(19) United States
(12) Patent Application Publication
(10) Pub. No.: US 2024/0207316 A1
(43) Pub. Date: Jun. 27, 2024

(54) CHIMERIC RECEPTOR FOR IMPROVING KILLING ACTIVITY OF IMMUNE CELLS AND APPLICATION THEREOF

(71) Applicant: ALLIFE MEDICINE (BEIJING) LIMITED, Beijing (CN)

(72) Inventors: Lida WU, Beijing (CN); Yuchun GU, Beijing (CN)

(73) Assignee: ALLIFE MEDICINE (BEIJING) LIMITED, Beijing (CN)

(21) Appl. No.: 18/516,502

(22) Filed: Nov. 21, 2023

Related U.S. Application Data
(63) Continuation of application No. PCT/CN2022/098848, filed on Jun. 15, 2022.

(30) Foreign Application Priority Data
Apr. 14, 2022 (CN) 202210386745.0

(51) Int. Cl. A61K 35/17; A61K 39/00; A61K 45/06; A61P 35/00; C07K 16/28; C12N 5/0783; C12N 15/86

(52) U.S. Cl. CPC A61K 35/17; A61K 39/001111; A61K 39/4613; A61K 39/4631; A61K 45/06; A61P 35/00; C07K 16/283; C12N 5/0646; C12N 15/86; A61K 2239/13; A61K 2239/17; A61K 2239/21; A61K 2239/22; A61K 2239/58; C12N 2740/15043

(57) ABSTRACT

The present invention belongs to the field of biological medicines, and particularly relates to a chimeric receptor for improving the killing activity of immune cells and an application thereof. Specifically, the present invention provides a fusion protein. The fusion protein comprises an extracellular part, an extracellular hinge region, a transmembrane region, and an intracellular region. Most preferably, the amino acid sequence of the fusion protein of the present invention is formed by sequentially connecting SEQ ID NO.: 1, SEQ ID NO.: 3, SEQ ID NO.: 5, SEQ ID NO.: 7, SEQ ID NO.: 9 and SEQ ID NO.: 11, and the immune cells expressing the fusion protein have strong killing activity.

### FIELD

The present invention belongs to the field of biomedicine and specifically relates to a chimeric receptor for improving killing activity of immune cell and application thereof.

### BACKGROUND

Due to the fact that NK cells (natural killer cells) have no MHC restrictions on their killing activity, they are called natural killer cells. Unlike T cells and B cells, NK cells do not require specific antigen sensitization stimulation to recognize and kill target cells. The killing effect of NK cells appears early after acting on target cells, and can be seen in vitro for 1 hour and in vivo for 4 hours.

The target cells of NK cells mainly include certain tumor cells, virus infected cells, certain self tissue cells (such as blood cells), parasites, etc. Therefore, NK cells are important immune factors for the body to fight against tumors and infections. The main mechanism of NK cell killing target cells is: 1. inducing cell lysis by releasing perforin and granzyme; 2. the activation pathway of apoptosis mediated by ligand induced receptors leads to apoptosis of target cells; 3. release cytokines (including NK cell cytotoxic factor and NK cell tumor necrosis factor) to kill target cells; 4. antibody dependent cell mediated cytotoxicity (ADCC).

When antibodies bind to tumor cell surface antigens through antigen binding sites and immune effector cell surface Fc receptors through Fc sites, immune effector cells are activated and kill tumor cells. This process is called antibody dependent cell mediated cytotoxicity (ADCC). There are three main types of Fc receptors: Fc γ RI (CD64), Fc γ RII (CD32), Fc γ RIII (CD16), of which the latter two can be further divided into: Fc γ RIIa, Fc γ RIIb, Fc γ RIIc, Fc γ RIIIa, Fc γ RIIIb. Different immune cells express specific Fc receptors, such as neutrophils, which typically express Fc γ RI, Fc γ RII, Fc γ RIIIb, while NK cells only express Fc γ RIIIa. Fc γ RIIIa is usually considered a key receptor that causes ADCC, so although NK cells, monocytes, macrophages, and neutrophils can all produce ADCC effects, NK cells are considered the most important cell population

The strength of ADCC is related to many factors, such as the affinity between antibodies and antigens, the affinity between antibodies and Fc receptors, the density of tumor antigens, the characteristics of tumor target cells, and the characteristics of immune effector cells. In general, the closer the bridge binding between tumor target cells and immune effector cells through antibodies, the stronger the ADCC effect. Therefore, antibodies with high affinity for antigens or Fc receptors mediate stronger ADCC effects. Tumor cells with high expression of target antigens are more sensitive to ADCC and are easily killed by the action of ADCC.

The degree of infiltration of NK cells at the tumor site also affects the effectiveness of immunotherapy. The recruitment of NK cells into the tumor can effectively improve the anti-tumor immune response. The chemokines and adhesion factors in the tumor microenvironment, by recruiting NK cells, promote an increase in NK infiltration in tumor tissue, which in turn enables NK cell surface activated receptors to recognize corresponding ligands on the tumor cell surface, release killing agents such as perforin, and exert anti-tumor cytotoxic effects.

NK cell therapy is a promising clinical research field, and research has confirmed its good safety and initial efficacy for certain cancer patients. NK cell therapy will play an important role in future tumor immunotherapy. With the increase of the incidence rate of cancer year by year, it is a common direction for doctors and patients to find a highly effective and non-toxic treatment. NK cell therapy can be used alone or in combination with other treatment methods for the treatment of various cancers, with high application prospects.

### SUMMARY

To further enhance the killing effect of NK cells, the present application provides a genetically modified NK cell, which has a stronger killing effect compared to ordinary NK cells; Furthermore, when used in combination with antibodies, the gene modified NK cells of the present invention recognize the Fc end of the antibody, recognize specific targets through the antibody, and have a strong killing effect on tumor cells.

The first aspect of the present invention provides a fusion protein, which comprises an extracellular portion, an extracellular hinge region, a transmembrane region, and an intracellular region; the extracellular part includes one or more of Ig-like C2 type 1 of CD16A, Ig-like C2 type 2 of CD16A, Ig-like C2 type 1 of CD64A, Ig-like C2 type 2 of CD64A, and Ig-like C2 type 3 of CD64A.

Preferably, the extracellular portion is any of the following:
1) Ig-like C2 type 1, Ig-like C2 type 2 of CD16a and Ig-like C2 type 3 of CD64 are sequentially connected in series;
2) Ig-like C2 type 1 of CD16A, Ig-like C2 type 2 of CD16A, Ig-like C2 type 1 of CD64A (CD64), Ig-like C2 type 2 of CD64A, and Ig-like C2 type 3 of CD64A are sequentially connected in series;
3) Ig-like C2 type 1 of CD64A, Ig-like C2 type 2 of CD64A, Ig-like C2 type 3 of CD64A, Ig-like C2 type 1 of CD16A, and Ig-like C2 type 2 of CD16A are sequentially connected in series;
4) Ig-like C2 type 1 of CD64A, Ig-like C2 type 2 of CD64A, Ig-like C2 type 3 of CD64A, and Ig-like C2 type 1 of CD16A are sequentially connected in series;
5) Ig-like C2 type 1 of CD64A, Ig-like C2 type 2 of CD64A, Ig-like C2 type 3 of CD64A, and Ig-like C2 type 2 of CD16A are sequentially connected in series.

Preferably, the extracellular hinge region (hinge region), transmembrane region, and intracellular region are the extracellular hinge region of CD64, the transmembrane region of CD16a, and the intracellular region of CD16a, respectively.

Preferably, the amino acid sequence of Ig-like C2 type 1 of CD16a is shown in SEQ ID NO.: 1 or has 1, 2, 3, 4, 5 or more mutations with the shown sequence.

Preferably, the amino acid sequence of the Ig-like C2 type 2 of CD16a is shown in SEQ ID NO.: 3 or has 1, 2, 3, 4, 5 or more mutations with the shown sequence.

Preferably, the amino acid sequence of the Ig-like C2 type 3 of CD64 is shown in SEQ ID NO.: 5 or has 1, 2, 3, 4, 5 or more mutations with the shown sequence.

Preferably, the amino acid sequence of the extracellular hinge region of the CD64 is shown in SEQ ID NO.: 7 or has 1, 2, 3, 4, 5 or more mutations with the shown sequence.

Preferably, the amino acid sequence of the transmembrane region of CD16a is shown in SEQ ID NO.: 9 or has 1, 2, 3, 4, 5 or more mutations with the shown sequence.

Preferably, the amino acid sequence of the intracellular region of CD16a is shown in SEQ ID NO.: 11 or has 1, 2, 3, 4, 5 or more mutations with the shown sequence.

Preferably, the amino acid sequence of the intracellular region of CD64A Ig-like C2 type 1 is shown in SEQ ID NO.: 15 or has 1, 2, 3, 4, 5 or more mutations with the sequence shown.

Preferably, the amino acid sequence of the intracellular region of CD64A Ig-like C2 type 2 is shown in SEQ ID NO.: 17 or has 1, 2, 3, 4, 5 or more mutations with the sequence shown.

Preferably, the fusion protein includes Ig-like C2 type 1 of CD16a, Ig-like C2 type 2 of CD16a, Ig-like C2 type 3 of CD64, extracellular hinge region of CD64, transmembrane region of CD16a, and intracellular region of CD16a.

The amino acid sequence of the fusion protein described in the present invention is sequentially connected by SEQ ID NO.: 1, 3, 5, 7, 9, and 11.

The “CD16a” mentioned in this invention is also known as “FcγR IIIA”, is an activated Fc receptor that, after being conjugated by the Fc region of the antibody, elicits signal transduction events that stimulate cells carrying the receptor to perform effector functions.

The fusion protein is also referred to as Chimeric-FcγR in the present invention.

On the other hand, the present invention also provides a nucleic acid encoding the fusion protein described in the present invention.

That is to say, the present invention provides a separated coding nucleic acid, which sequentially encodes Ig-like C2 type 1 of CD16a, Ig-like C2 type 2 of CD16a, Ig-like C2 type 3 of CD64, extracellular hinge region of CD64, transmembrane region of CD16a, and intracellular region of CD16a.

Preferably, the coding nucleic acid sequence of Ig-like C2 type 1 of CD16a is shown in SEQ ID NO.: 2.

Preferably, the coding nucleic acid sequence of Ig-like C2 type 2 of CD16a is shown in SEQ ID NO.: 4.

Preferably, the coding nucleic acid sequence of the Ig-like C2 type 3 of the CD64 is shown in SEQ ID NO.: 6.

Preferably, the coding nucleic acid sequence of the extracellular hinge region is shown in SEQ ID NO.: 8.

Preferably, the coding nucleic acid sequence of the transmembrane region is shown in SEQ ID NO.: 10.

Preferably, the coding nucleic acid sequence of the intracellular region is shown in SEQ ID NO.: 12.

Preferably, the coding nucleic acid sequence of CD64A Ig-like C2 type 1 is shown in SEQ ID NO.: 16.

Preferably, the coding nucleic acid sequence of CD64A Ig-like C2 type2 is shown in SEQ ID NO.: 18.

Preferably, the nucleic acid encoding the Chimeric-Fc γ R according to the present invention is a DNA sequence constructed by SEQ ID NO.: 2, 4, 6, 8, 10, and 12 connected sequentially.

On the other hand, the present invention also provides an expression vector that expresses the aforementioned fusion protein or contains the encoding nucleic acid.

The term “expression vector” refers to well-known bacterial plasmids, bacteriophages, yeast plasmids, plant cell viruses, mammalian cell viruses such as adenoviruses, retroviruses, lentiviruses, or other vectors in the art.

In short, as long as it can replicate and stabilize in the host body, any plasmid and vector can be used. An important feature of expression vectors is that they typically contain replication starting points, promoters, marker genes, and translation control elements.

The exemplary embodiment of the present invention utilizes the pLV-EF1a-IRES-Hygro lentivirus vector.

On the other hand, the present invention also provides host cells containing or expressing one or more of the aforementioned fusion proteins, encoding nucleic acids, and vectors.

Preferably, the host cells are human immune cells and stem cells.

More preferably, the host cells are NK cells or iPSCs (induced pluripotent stem cells) that can be induced into NK cells.

Preferably, the host cells include autologous cells or allogeneic cells.

Preferably, the host cells can be mature commercial cell line products or obtained through in vitro culture.

The host cell of the present invention can also be a prokaryotic cell, such as a bacterial cell; or lower level eukaryotic cells, such as yeast cells; or higher eukaryotic cells, such as mammalian cells. Representative examples include: Escherichia coli, Streptomyces genus; Bacterial cells of Salmonella typhimurium; Fungal cells such as yeast; Plant cells; CHO, COS, 293 cells, etc.

On the other hand, the present invention also provides a method for preparing highly cytotoxic immune cells, which includes introducing one or more of the fusion protein, coding nucleic acid, or vector into immune cells, or introducing one or more of the fusion protein, coding nucleic acid, or vector into stem cells, and then inducing differentiation into immune cells.

Preferably, the immune cells include one or more of T cells, B cells, K cells, and NK cells.

Preferably, the immune cells are NK cells.

As used in the application, the term “killing activity” when used to describe the activity of immune cells such as NK cells involves killing target cells through any of various biological, biochemical, or biophysical mechanisms.

Preferably, the stem cells are induced pluripotent stem cells (iPSCs): pluripotent stem cells with the potential to differentiate into multiple cells obtained by transferring pluripotent factors into adult cells and reprogramming the initial genome expression profile. And iNK (iPSC derived NK) is a natural killer cell induced by iPSC differentiation.

Preferably, the immune cells include autologous immune cells and allogeneic immune cells.

Preferably, the stem cells include autologous stem cells and allogeneic stem cells.

The introduction of one or more of the aforementioned fusion proteins, coding nucleic acids, and vectors into immune cells can be achieved through various techniques known to those skilled in the art. These techniques include, but are not limited to, electrophoresis and electroporation, protoplast fusion, calcium phosphate precipitation, cell fusion using encapsulated DNA, microinjection, and complete virus transfection.

As used in the specific embodiments of the present invention, the virus vector is introduced into the host cell through lentivirus transfection technology to obtain a host cell that stably expresses the fusion protein, which represents the presence of the encoding nucleic acid and the vector where the encoding nucleic acid is located in the host cell.

On the other hand, the present invention also provides a pharmaceutical composition comprising one or more of the aforementioned host cells, fusion proteins, encoding nucleic acids, and vectors.

The pharmaceutical composition also contains other drugs for treating cancer or structure that is recognized by Chimeric-FcγR.

More preferably, the drug is a monoclonal antibody drug.

Exemplarily, the monoclonal antibody drugs include drugs that have already been marketed, such as Matuximab, Trastuzumab, Cetuzumab, Dalizumab, Tanizumab, Abavozumab, Admuzumab, Aftuzumab, Alenmumab, Peihua Azzumab, Amatuzumab, Abazumab, Paviximab, Betomozumab, Belimumab, Bevaczumab, Mo Bivaczumab Berentzumab Vitilin, Mocantuzumab, Lacanthuzumab, Carozumab Penditide, Catuxumab, Poxituzumab, Situxumab, Kenazumab, Daxizumab, Dalozumab, Demozumab, Emeximab, Ezuzumab, Ezuzumab, Ensiximab, Epacizumab, Ermazumab, Adazumab, Farazumab, Fentolumab, Galiximab Gistuzumab, Gistuzumab, Giriximab, Gleizumab Vititin, Teimozumab, Igovozumab, Laindacizumab, Intimazumab, Izuzuzumab, Ozomicin, Ipimazumab, Itumazumab, Labezzumab, Lexazumab, Lintuzumab, Molovozumab, including their antigen-binding fragments;

The monoclonal antibody drug can also be a commercialized, clinically unproven monoclonal antibody product, as verified by the specific embodiment of the present invention, and the antibody (FOLH1/3734) with product number ab268061 provided by abcam company.

Preferably, the pharmaceutical composition also includes pharmaceutically acceptable carriers, diluents, or excipients.

Preferably, the pharmaceutically acceptable carriers, diluents, or excipients include, but are not limited to, any adjuvants, vectors, excipients, flow aids, sweeteners, diluents, preservatives, dyes/colorants, flavor enhancers, surfactants, wetting agents, dispersants, suspensions, stabilizers, isotopes, solvents, surfactants or emulsifiers that have been approved by the Food and Drug Administration or the China Food and Drug Administration for use in humans or livestock.

Preferably, the pharmacutical composition can be tablets, pills, powders, granules, capsules, tablets, syrups, liquids, emulsions, suspensions, controlled release preparations, aerosols, films, injections, intravenous drops, transdermal absorption preparations, ointments, lotions, adhesive preparations, suppositories, small pills, nasal preparations, lung preparations, eye drops, etc., oral or parenteral preparations.

On the other hand, the present invention provides a method for killing target cells in vitro, which includes contacting the target cells with one or more of the aforementioned fusion proteins, polynucleotides, vectors, host cells, and pharmaceutical compositions;

Preferably, the target cell is a cancer cell; the cancer cells include the following cancer cells: cervical cancer, seminoma, testicular lymphoma, prostate cancer, ovarian cancer, lung cancer, rectal cancer, breast cancer, skin squamous cell cancer, colon cancer, liver cancer, pancreatic cancer, gastric cancer, esophageal cancer, thyroid cancer, bladder transitional cell cancer, leukemia, brain tumor, gastric cancer, peritoneal cancer, head and neck cancer, endometrial cancer, kidney cancer, female genital tract cancer, carcinoma in situ Neurofibroma, bone cancer, skin cancer, gastrointestinal stromal tumor, mast cell tumor, multiple myeloma, melanoma, glioma;

Preferably, the target cells are prostate cancer cells.

On the other hand, the present invention also provides a method for treating diseases, which includes administering one or more of the aforementioned drug compositions, host cells, fusion proteins, encoded nucleic acids, and vectors.

The pharmaceutical composition described in this article can be administered using various well-known methods in the art. The administration may include, for example, the following methods: oral ingestion, direct injection (such as systemic or stereotactic), and the pharmaceutical composition may also be modified into a biomaterial that can release cells, such as polymer matrix, gel, osmotic membrane, permeability system, multi-layer coating, particles, implantable matrix device, micro osmotic pump, implantation pump, injectable gel and hydrogel, liposome micelle (e.g. up to 30 μm), nanospheres (e.g. less than 1 μm), microspheres (e.g. 1-100 μm), or other suitable delivery media to provide the required release rate in different proportions. Other methods for controlling the release and delivery of drug compositions are known to technical personnel and are within the scope of this disclosure.

In the context of the present invention, the terms “treatment”, “therapy”, etc., within the scope of any of the diseases referred to in this article, mean alleviating or alleviating at least one symptom associated with such a disease, or slowing or reversing the progression of such a disease. In the meaning of the present invention, the term “treatment” also refers to inhibiting, delaying the onset of the disease (i.e., the period before the clinical manifestation of the disease), and/or reducing the risk of disease development or deterioration. For example, the term “treatment” related to cancer can refer to eliminating or reducing a patient's tumor burden, or preventing, delaying, or inhibiting metastasis.

On the other hand, the present invention also provides the application of the aforementioned pharmaceutical compositions, host cells, fusion proteins, encoding nucleic acids, and vectors in the preparation of cancer immunotherapy drugs, autoimmune disease drugs, anti-aging drugs, medical beauty products, and metabolic disease drugs.

The cancer described in the present invention can be a blood cancer or a cancer with a solid tumor. Preferably, the cancers include cervical cancer, seminoma, testicular lymphoma, prostate cancer, ovarian cancer, lung cancer, rectal cancer, breast cancer, skin squamous cell cancer, colon cancer, liver cancer, pancreatic cancer, stomach cancer, esophageal cancer, thyroid cancer, bladder transitional epithelial cancer, leukemia, brain tumor, stomach cancer, peritoneal cancer, head and neck cancer, endometrial cancer, kidney cancer, female genital tract cancer, carcinoma in situ, neurofibroma, bone cancer Skin cancer, gastrointestinal stromal tumor, mast cell tumor, multiple myeloma, melanoma, glioma;

Examples of autoimmune diseases described in the present invention include achalasia of the cardia; Addison's disease; Adult Steele's disease; No gammaglobulinemia; Alopecia areata; Amyloidosis; Ankylosing spondylitis; Anti GBM/anti TBM nephritis; Anti phospholipid syndrome; Autoimmune vascular edema; Autoimmune autonomic dysfunction; Autoimmune encephalomyelitis; Autoimmune hepatitis; Autoimmune inner ear disease (AIED); Autoimmune myocarditis; Autoimmune ovarian inflammation; Autoimmune orchitis; Autoimmune pancreatitis; Autoimmune retinopathy; Autoimmune urticaria.

Examples of metabolic diseases of the invention include diabetes, diabetes ketoacidosis, hyperglycemia and hypertonic syndrome, hypoglycemia, gout, protein energy malnutrition, vitamin A deficiency, scurvy, vitamin D deficiency, and osteoporosis. The metabolic diseases known to technical personnel in this field are diseases caused by metabolic problems, including metabolic disorders and metabolic exuberance.

On the other hand, the present invention also provides the application of the drug composition, host cells, fusion proteins, coding nucleic acids, and vectors in improving the therapeutic effect of monoclonal antibodies.

More preferably, the application is to enhance the application of PSMAmAb antibody (manufacturer abcam, product number ab268061) in killing LNCaP cells (human prostate cancer cells).

According to the specific embodiment of the present invention, the killing activity of NK cells expressing the fusion protein described in the present invention against cancer cells was verified through prostate cancer cell LNCaP.

### BRIEF DESCRIPTION OF DRAWINGS

FIG. 1 shows the structure diagram of Chimeric-FcγR as described in the present invention.

FIG. 2 shows the structure diagram of each variant of Chimeric-FcγR.

FIG. 3 shows effect verification of Chimeric-Fc γ R and its variants.

FIG. 4 shows identification result diagram of expression level of Chimeric-FcγR or mutCD16A on the prepared iPSC.

FIG. 5 shows the statistical results of the percentage of positive NK cells after activation of NK cells.

FIG. 6 shows the statistical results of the killing activity of different groups of drugs on LNCaP cancer cell lines.

FIG. 7 shows the statistical results of the effects of different groups of drugs on tumor weight in mice in the experiment.

### DETAILED DESCRIPTION

The following is a further explanation of the present invention in conjunction with embodiments. The following is only a preferred embodiment of the present invention and does not impose any other form of limitation on the present invention. Any technical personnel familiar with the profession may use the disclosed technical content to modify it into equivalent embodiments with the same changes. Any simple modifications or equivalent changes made to the following embodiments based on the technical essence of the present invention without departing from the content of the present invention scheme shall fall within the scope of protection of the present invention.

### Example 1: Vector Construction of Chimeric-Fcγ R and Its Variants, Lentivirus Packaging, Stable Transfection of NK Cells, and Identification of ADCC Effect

### Vector Construction

### 1. Experimental Materials

Skeleton vector: pLV-EF1a-IRES-Hygro plasmid (addgene, product number Plasmid #85134)

### 2. Experimental Methods

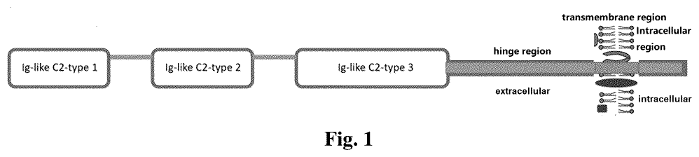

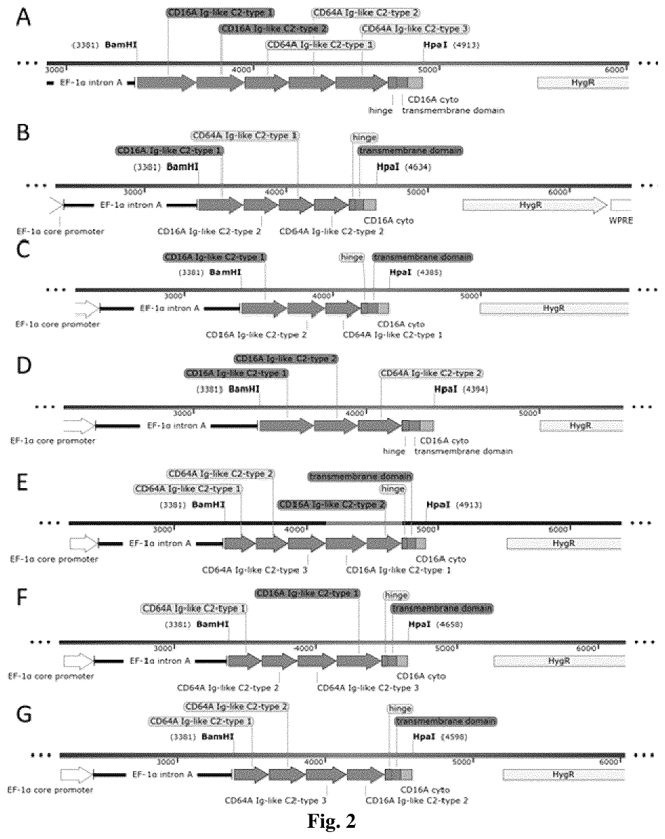

1. Using pLV-EF1a-IRES-Hygro plasmid (addgene, product number Plasmid #85134) as the skeleton vector of Chimeric-FcγR and its variants, with the cleavage sites EcoRI and Hpa1;
2. The structure of Chimeric-FcγR is shown in FIG. 1, and the extracellular portion includes Ig-like C2 type 1, Ig-like C2 type 2 of CD16a, Ig-like C2 type 3 of CD64, Chimeric-FcγR also includes the hinge region of CD64A, the transmembrane region of CD16a, and the intracellular region of CD16a.
3. The structure of each variant of Chimeric-FcγR is shown in FIG. 2, the extracellular parts of Chimeric-FcγR and its variants are summarized in the table below, the hinge region, transmembrane region, intracellular region are consistent with those of Chimeric FcγR.

| | CD16A Ig-like | CD16A Ig-like | CD64A Ig-like | CD64A Ig-like | CD64A Ig-like |
|---|---|---|---|---|---|
| | C2-type 1 | C2-type 2 | C2-type 1 | C2-type 2 | C2-type 3 |
| Amino Acid | SEQ ID NO.: | SEQ ID NO.: | SEQ ID NO.: | SEQ ID NO.: | SEQ ID NO.: |
| Sequence | 1 | 3 | 15 | 17 | 5 |
| Nucleotide | SEQ ID NO.: | SEQ ID NO.: | SEQ ID NO.: | SEQ ID NO.: | SEQ ID NO.: |
| sequence | 2 | 4 | 16 | 18 | 6 |
| Chimeric- | + | + | − | − | + |
| FcγR | | | | | |
| Variant A | + | + | + | + | + |
| Variant B | + | + | + | + | - |
| Variant C | + | + | + | - | - |
| Variant D | + | + | - | + | - |
| | CD64A Ig-like | CD64A Ig-like | CD64A Ig-like | CD16A Ig-like | CD16A Ig-like |
| | C2-type 1 | C2-type 2 | C2-type 3 | C2-type 1 | C2-type 2 |
| Amino Acid | SEQ ID NO.: | SEQ ID NO.: | SEQ ID NO.: | SEQ ID NO.: | SEQ ID NO.: |
| Sequence | 15 | 17 | 5 | 1 | 3 |
| Nucleotide | SEQ ID NO.: | SEQ ID NO.: | SEQ ID NO.: | SEQ ID NO.: | SEQ ID NO.: |
| sequence | 16 | 18 | 6 | 2 | 4 |
| Variant E | + | + | + | + | + |
| Variant F | + | + | + | + | - |
| Variant G | + | + | + | - | + |

Explanation: + represents the existence of the structure, − represents the absence of the structure

4. The gene synthesis of each sequence in the above table is carried out by Anhui General Biotechnology Co., Ltd.
5. Chimeric-Fcγ R and its variants were inserted between EcoRI and Hpa1 of the pLV-EF1a-IRES-Hygro plasmid, respectively, to obtain the vectors.

### Lentivirus Packaging

### 1. Experimental Materials

| Name | Company | Article number |
|---|---|---|
| FBS | hyclone | SH30084.03 |
| DMEM | Thermo Fisher Scientific | 11965084 |
| PEI | POLYSCIENCES | 23966 |
| pMD2.G | addgene | #12259 |
| pCMV-VSVG | addgene | #8454 |
| pRSV-Rev | addgene | #12253 |
| Lenti-XTM Concentrator | clonetech | 631232 |

### 2. Experimental Methods

1. Cell inoculation: 10 cm disc inoculation 1.5×10⁷ 293T cells. Added 10 ml of DMEM culture medium containing 10% FBS, incubated overnight at 37° C.in a 5% CO2 incubator, and transfected after 16-24 hours.
2. Cell transfection: the intersection of cell growth reached 80-90%, ready for transfection. The transfection system is as follows:

| A solution | | | B solution | | |
|---|---|---|---|---|---|
| Each vector | 6.65 | μg | PEI | 45 | μg |
| pMD2.G | 4.3 | μg | DMEM | 500 | μl |

| | | |
|---|---|---|
| pCMV-VSVG ( | 2.3 | μg |
| pRSV-Rev (addgene, #12253) | 1.68 | μg |
| Serum free DMEM | 500 | μl |

Added B solution dropwise to A solution, shook well while adding, and let stand at room temperature of 22-26° C.for 15 minutes. Added drop by drop to the culture dish, gently shook well, 5% CO₂, and incubated overnight at 37° C.
3. Transfection medium changing: after 16-18 hours, removed the culture medium containing the transfection reagent, added 10 ml of DMEM containing 10% FBS, continue cultivation at 5% CO2 and 37° C.
4. The first harvest of the virus: 48 hours after transfection, the cell supernatant was harvested and transferred to a 50 ml centrifuge tube, centrifuged at 3000 rpm for 10 minutes, and the supernatant was filtered by using a 0.45 μm filter membrane and stored at 4° C. Added 10 ml of DMEM containing 10% FBS to the cells, continue to culture at 5% CO2 and 37° C.
5. Second harvest of the virus: harvest the cell supernatant, transferred it to a 50 ml centrifuge tube, centrifuged at 3000 rpm for 10 minutes, the supernatant was filtered by using a 0.45 μm filter membrane and stored at 4° C. Cells were treated with 10% disinfectant (84 disinfectant) and discarded.
6. Virus concentration: the collected lentivirus components were filtered using a 0.45 μm filter membrane to remove bacterial contamination. Mixed the filtered components with Lenti XTM Concentrator in a volume ratio of 3:1, gently inverted and mixed well.
7. Incubated at 4° C.for 30 minutes or overnight.
8. Centrifuged at 4° C. 1500 g for 45 minutes, and white precipitates was seen at the bottom of the tube after centrifugation.
9. Carefully removed the supernatant and avoid damaging the white precipitate.
10. Resuspension the precipitate with an appropriate volume of lentivirus preservation solution, and separated and stored the obtained lentivirus at −80° C.

### Cell Killing Tsest

### 1. Experimental Materials

| | | Article |
|---|---|---|
| Name | Company | number |
| LNCaP Cell | Procell Life | CL-0143 |
| (Human prostate cancer) | Science&Technology Co., Ltd. | |
| Caspase-3/7 Green Apoptosis | Essen Bioscience | 4440 |
| Assay Reagent | | |
| PSMAmAb antibody | abcam | ab268061 |

### 2. Experimental Methods

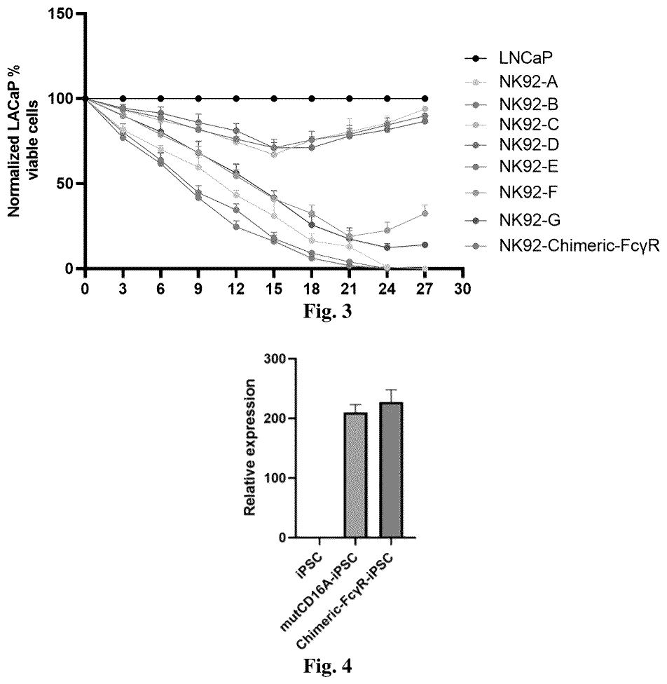

Obtaining lentivirus lenti-A, lenti-B, lenti-C, lenti-D, lenti-E, lenti-F, lentiG, and lenti-Chimeric-FcγR expressing the above structures through lentivirus packaging. Infected the NK92 cell line with the above viruses separately, and screen for 7-14 days using Hexadimethyrine bromide to obtain positive cell lines, named NK92-A, NK92-B, NK92-C, NK92-D, NK92-E, NK92-F, NK92-G, and NK-Chimeric-FcγR, respectively.
1. Spread LNCaP cells onto 96 well plates with 4000 cells per well and incubated for 24 hours.
2. After 24 hours, collected LNCaP cells from 3 wells, count them, and calculated the average value.
3. After counting, incubated LNCaP cells from 96 well plates with Caspase-3/7 Green Apoptosis Assay Reagent for 30 minutes.
4. According to the count, separate NK92-A, NK92-B, NK92-C, NK92-D, NK92-E, NK92-F, NK92-G, and NK-Chimeric-FcγR cells and tumor cells were inoculated into a 96 well plate with a 1:1 target ratio, and 1 μg/mL of PSMAmAb was added to the culture medium, which was cultured in a 37 degree cell incubator.
5. Analyzed every 3 hours. The results were shown in FIG. 3.

### 3. Explanation of Experimental Results

1. The ADCC effect of NK92-A and NK92-E is better than other variants, indicating that the three Ig-like C2 structures of CD64A are intact, which is more conducive to the ADCC effect of NK cells. The effect of NK92-E is better than that of NK92-A, indicating that the IgG Fc binding domain of CD64A is further away from the cell membrane structure, which is conducive to its ADCC effect.
2. Comparison of NK92-A, NK92-B, NK92-C, NK92-D, and NK92-Chimeric-FcγR showed that, the ADCC effect of NK92-A and NK92-Chimeric-FcγR is better, indicating that the Ig-like C2 type 3 structure of CD64A is more conducive to NK's ADCC effect compared to the other two structures.
3. Compared with NK92-E, NK92-F, and NK92-G, NK92-E has a better ADCC effect, indicating that connecting one structure after the Ig-like C2 type 3 structure of CD64A will affect the ADCC effect of NK92. However, connecting two complete structures of CD16A will not affect its ADCC effect.
4. The ADCC effect of NK92-E and NK92-Chimeric-FcγR is basically unchanged, indicating that the Ig-like C2 type 1 and Ig-like C2 type 2 structures of CD16A are similar to the Ig-like C2 type 1 and Ig-like C2 type 2 structures of CD64A. The Ig-like C2 type 3 of CD64A can enhance the ADCC effect of NK92.

According to the analysis of the results in FIG. 3, the structure of Chimeric-FcγR endows NK cells with the strongest ADCC effect, further investigating the NK cells expressing Chimeric-FcγR.

### Example 2: Preparation of Chimeric-FcγR-iNK From iPSC Source and Its Stability Detection Under Activation Conditions

Constructed Chimeric-FcγR as described in Example 1, and constructed mutCD16A (amino acid sequence such as SEQ ID NO.: 14, nucleotide sequence such as SEQ ID NO.: 13) as a control to further validate the characteristics of the Chimeric-FcγR of the present invention.

### Chimeric-FcγR-iPSC Cells Stable Transformation and Screening

### 1. Experimental Materials

| Name | Company | Article number |
|---|---|---|
| Puromycin | Merck | P9620 |
| mTeSR1 | Stem cell technologies | #85850 |
| Dispase II | Merck | D4693 |
| Hexadimethrine bromide | Merck | H9268 |

### 2. Experimental Methods

1. On day 0, approximately 24 hours before initial transduction, when the cell density reached about 80%, 1 mg/mL Dispase II was used for cell passage.
2. Inoculated iPSC cells in a 1:3 ratio into a 24 well culture plate, taking care to maintain cell clusters with a diameter of approximately 50-60 μM.
3. On the first day, used 500 μL preheated (37° C.) mTeSR1 incubated cells, with 6 μg/mL Hexadimethrine bromide added to the culture medium, incubated the cells in an incubator for 15 minutes.
4. Infected iPSC cells by 10 μL of 1×10⁶ TU/mL virus particles per hole. Incubated at 37° C., 5% CO₂, and 95% humidity for 18-20 hours.
5. On the second day, removed the culture medium and added 500 μL preheated (37° C.) mTeSR1 incubated cells, added 6 μg/mL Hexadimethrine bromide to the culture medium, incubated the cells in an incubator for 15 minutes.
6. Added three times the initial amount of virus particles (starting from day 1) to the culture medium. I.e. 30 μL of 1×10⁶ TU/mL virus particles were secondarily infected. Incubated at 37° C., 5% CO₂, and 95% humidity for 18-20 hours.
7. On the 3rd and 4th days, removed the culture medium daily and replaced it with 500 μL of preheated medium without Hexadimethyrine bromide.
8. On days 5-8, used 500 μL preheated medium to replace the solution daily and added 1 μg/mL purinomycin to the medium.
9. Continuously used 1 μg/mL purinomycin to screen positive cells until the cells were stable.
10. The stably transformed cell lines were named NK92 Chimeric-FcγR-iPSC (Chimeric-FcγR-iPSC), mutCD16A-iPSC, respectively.

### Identification of Chimeric-FcγR-iPSC Stable Transgenic Cells

### 1. Experimental Materials

| Name | Company | Article number |
|---|---|---|
| TRIZOL | sigma | T9424 |
| PerfectStart ® Green qPCR SuperMix | TransGen | AQ601-02 |
| | Biotech | |

### 2. Experimental Methods

1. Collected 200W Chimeric-FcγR-iPSC cells, mutCD16A-iPSC cells, added 1 ml of TRIZOL, extracted RNA and measured RNA concentration. Took 1 μg RNA and was inverted into cDNA and pre mixed according to the following table system.

| Component | Volume | |
|---|---|---|
| Forward Primer (10 μm) | 0.4 | μl |
| Reverse Primer (10 μm) | 0.4 | μl |
| 2×TransStar Top/Tip Green qPCR | 10 | μl |
| SuperMix | | |
| Nuclease-free Water | 7.2 | μl |
| cDNA | 2 | μl |
| Total volume | 20 | μl |

2. Then, the above system was placed in a Light cycle instrument and reacted in a 3-step method with a cycle number of 45. The reaction system is as follows:

| Temp | Time | |
|---|---|---|
| 94° C | 30 s | |
| 94° C. | 5 s | 45 cycles |
| 55° C. | 15 s | |
| 72° C. | 10 s | |

The detection primer sequence is as follows

### Chimeric-FcγR Structural Detection Primers:

Forword primer:
ACTCAAAGACAGCGGCTCCTA
Reverse primer:
ACAGCTCAGGGTGACCAGATT

### MutCD16A Structural Detection Primers:

Forword primer:
CCTCCTGTCTAGTCGGTTTGG
Reverse primer:
TCGAGCACCCTGTACCATTGA

### 3. Experimental Results

As shown in FIG. 4, detected the expression level of Chimeric-FcγR or MutCD16A, to determine pLV-EF1a-Chimeric-FcγR-IRES-Hygro or pLV-EF1a-mutCD16A-IRES-Hygro vectors have been successfully expressed.

### Chimeric-FcγR Stability Testing

### 1. Experimental Materials

| | | Article |
|---|---|---|
| Name | Company | number |
| STEMdiff ™ NK Cell Kit | Stem cell technologies | #100-0170 |
| PMA/Ionomycin mixture (250×) | MultiSciences | 70-CS1001 |
| DPBS | Thermo Fisher Scientific | 14190144 |
| Anti-Human CD16 | BD Biosciences | 560995 |
| K-562 | Wuhan Procell Life | CL-0130 |
| | Science&Technology | |
| | Co., Ltd. | |
| Mitomycin C | Sigma | M5353 |

### 2. Experimental Methods

1. Used STEMdiff™ NK Cell Kit, iPSC, Chimeric-FcγR-iPSC and mutCD16A-iPSC were differentiated into iNK cells, resulting in iNK cells were named iNK, Chimeric-FcγR-iNK and mutCD16A-iNK, respectively.
2. Took 2 million iNK, Chimeric-FcγR-iNK and mutCD16A-iNK, used 1-fold PMA/Ionomycin mixture (250×) to stimulate cell for 4 hours. Alternatively, K562 cells treated with an equal proportion of mitomycin C can be treated with iNK and Chimeric-Fc, respectively γ Incubate R-iNK and mutCD16A-iNK for 4 hours. At the same time, a blank control was set up without any stimulation of NK cells mentioned above.
3. After cell activation, cleaned the cells twice with DPBS and resuspended them at 100 μL DPBS with 2% FBS, the cells were incubated with Anti Human CD16 for 1 hour according to the manufacturer's instructions.
4. After incubation, cleaned the cells twice with DPBS and resuspended them at 100 μL DPBS with 2% FBS, flow cytometry analysis was performed on the aforementioned cells.

### 3. Experimental Results

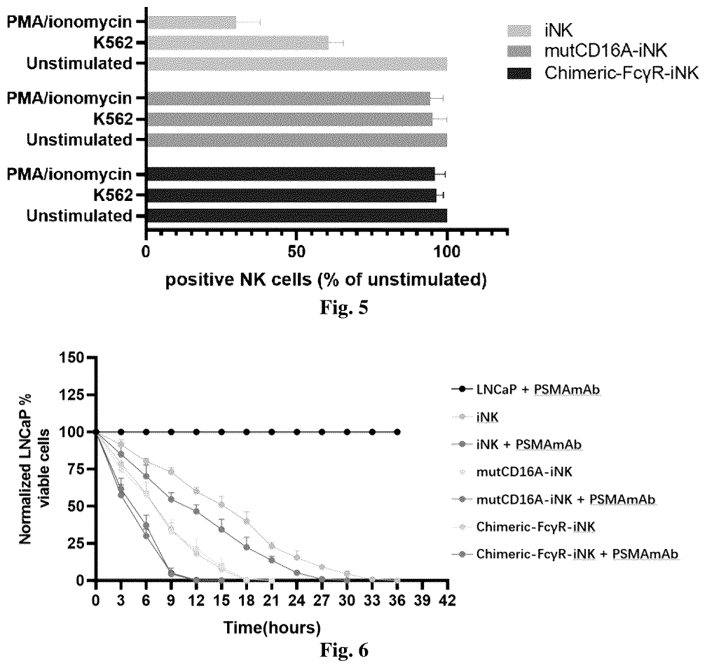

K562 (treated with mitomycin C) and PMA/lonomycin were used to activate NK cells and detect the percentage of activated NK cells. When NK cells were activated, unmodified CD16A will be removed by metalloproteinase (ADAM17). The experimental results showed a significant decrease in the percentage of unmodified iNK cells after detecting the percentage of NK cells. And mutCD16A-iNK cells and Chimeric-FcγR-iNK cells after cell activation, mutCD16A and Chimeric-FcγR protein were not cleaved by metalloenzymes, so the proportion of iNK positive cells was still at a high level (as shown in FIG. 5).

Therefore, it is inferred that Chimeric-FcγR-iNK of the present invention has better killing activity than unmodified iNK cells, so we will continue to compare the killing effect on tumor cells of Chimeric-FcγR-iNK and mutCD16A at the cellular and animal experimental levels as follows.

### Cell Killing Test

### 1. Experimental Methods

1. Spread LNCaP cells onto 96 well plates with 4000 cells per well and incubated for 24 hours.
2. After 24 hours, collected LNCaP cells from 3 wells, count them, and calculated the average value.
3. After counting, incubated LNCaP cells from 96 well plates with Caspase-3/7 Green Apoptosis Assay Reagent for 30 minutes.
4. According to the count, INK, Chimeric-FcγR-iNK and mutCD16A-iNK cells and tumor cells were inoculated into a 96 well plate with a 1:1 target ratio, and the groups shown in the table were set separately, and cultured in an IncuCyt incubator.

| | | | | PSMAmAb |
|---|---|---|---|---|
| | Group | LNCaP | NK cells | antibody |
| Control | LNCaP + PSMAmAb | + | − | + |
| group | | | | |
| Without | iNK | + | + | − |
| antibody | mutCD16A-iNK | + | + | − |
| | Chimeric-FcγR-iNK | + | + | − |
| With | iNK + PSMAmAb | + | + | + |
| antibody | mutCD16A-iNK + | + | + | + |
| | PSMAmAb | | | |
| | Chimeric-FcyR-iNK + | + | + | + |
| | PSMAmAb | | | |

Explanation: + indicating with the addition of cells or antibody, − indicating without the addition of cells or antibody cells or antibody

5. Afterwards, took photos and recorded every 3 hours for analysis, and the statistical results are shown in FIG. 6.

### 2. Experimental Results

1. Comparison of MutCD16A-iNK, Chimeric-FcγR-iNK and iNK+PSMAmAb groups, mutCD16A-iNK and Chimeric-FcγR-iNK groups have a much stronger killing effect on LNCaP cells, indicating that mutCD16A iNK and Chimeric FcγR of MutCD16A, Chimeric-FcγR-iNK in the group can enhance NK's killing ability.
2. The comparison between the iNK group and the iNK+PSMAmAb group showed that the iNK+PSMAmAb group had a stronger killing effect on LNCaP cells, and iNK had a certain ADCC effect.
3. The comparison between the mutCD16A iNK and mutCD16A iNK+PSMAmAb groups showed that the mutCD16A iNK+PSMAmAb group had stronger cytotoxicity to LNCaP cells, and mutCD16A iNK+PSMAmAb had stronger ADCC effect.
4. Comparison of Chimeric-FcγR-iNK and Chimeric-FcγR-iNK+PSMAmAb group shows that Chimeric-FcγR-iNK+PSMAmAb group has strong cytotoxicity against LNCaP cells, while Chimeric-FcγR-iNK+PSMAmAb has a stronger ADCC effect.
5. Comparison of MutCD16A-iNK+PSMAmAb and Chimeric-FcγR-iNK+PSMAmAb group shows that Chimeric-FcγR-iNK+PSMAmAb group showed slightly stronger cytotoxicity to LNCaP cells, while Chimeric-FcγR-iNK+PSMAmAb has a stronger ADCC effect.

In summary, the experimental results of killing LNCaP cells indicate that NK cells expressing the fusion protein described in the present invention have superior killing activity compared to unmodified NK cells, regardless of the presence or absence of antibodies.

### Example 3: In Vivo Killing Experiment

### 1. Experimental Materials

| Name | Company | Article number |
|---|---|---|
| C42 Cells | ATCC | CRL-3314 |
| (human prostate cancer cells) | | |
| NOD SCID mice | Vital River | 406 |

### 2. Experimental Methods

1. Subcutaneous injection of NOD SCID mice 1×10⁶ fluorescent labeled C42 cells formed visible tumors 3 weeks later.
2. After the formation of the tumor model, it is first analyzed through a small animal imaging system, and then different groups are injected through the tail vein, with injections each 5×10⁶ INK, iNK+100 μg PSMAmAb mutCD16A-iNK+100 μg PSMAmAb, Chimeric-FcγR-iNK+100 μg PSMAmAb. Simultaneously setting non treated controls
3. After injecting NK, the tumor status was analyzed weekly through a small animal imaging system.
4. After the experiment, dissect the animals and separate the tumor tissue for weighing.

### 3. Experimental Results

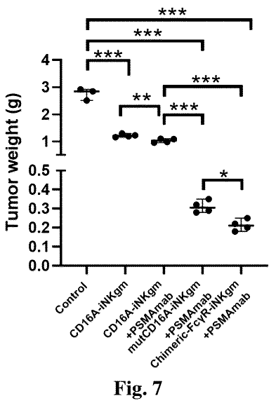

The statistical analysis of tumor weight after the experiment was shown in FIG. 7. Explanation of experimental results:
1. The comparison between the iNK group and the iNK+PSMAmAb group showed that the iNK+PSMAmAb group had a stronger ADCC effect and a stronger tumor killing effect.
2. Comparison of iNK+PSMAmAb, mutCD16A-iNK+PSMAmAb, Chimeric-FcγR-iNK+PSMAmAb group showed, mutCD16A iNK+PSMAmAb, Chimeric-FcγR-iNK+PSMAmAb group has a stronger ADCC effect and stronger tumor killing effect.
3. Comparison of mutCD16A-iNK+PSMAmAb and Chimeric-FcγR-iNK+PSMAmAb group showed, Chimeric-FcγR-iNK+PSMAmAb group has a stronger ADCC effect and stronger tumor killing effect.

1. A fusion protein, comprising an extracellular portion, an extracellular hinge region, a transmembrane region, and an intracellular portion, wherein the extracellular portion is any of the following:
1. Ig-like C2 type 1, Ig-like C2 type 2 of CD16a and Ig-like C2 type 3 of CD64 are sequentially connected in series;
2. Ig-like C2 type 1 of CD16A, Ig-like C2 type 2 of CD16A, Ig-like C2 type 1 of CD64A, Ig-like C2 type 2 of CD64A, and Ig-like C2 type 3 of CD64A are sequentially connected in series;
3. Ig-like C2 type 1 of CD64A, Ig-like C2 type 2 of CD64A, Ig-like C2 type 3 of CD64A, Ig-like C2 type 1 of CD16A, and Ig-like C2 type 2 of CD16A are sequentially connected in series;
4. Ig-like C2 type 1 of CD64A, Ig-like C2 type 2 of CD64A, Ig-like C2 type 3 of CD64A, and Ig-like C2 type 1 of CD16A are sequentially connected in series; and
5. Ig-like C2 type 1 of CD64A, Ig-like C2 type 2 of CD64A, Ig-like C2 type 3 of CD64A, and Ig-like C2 type 2 of CD16A are sequentially connected in series; and
wherein the extracellular hinge region, the transmembrane region, and the intracellular region are respectively the extracellular hinge region of CD64, the transmembrane region of CD16a, and the intracellular region of CD16a.

2. The fusion protein as claimed in claim 1, wherein:
the amino acid sequence of Ig-like C2 type 1 of CD16a is shown in SEQ ID NO.: 1 or has 1, 2, 3, 4, 5 or more mutations compared to the sequence shown,
the amino acid sequence of Ig-like C2 type 2 of CD16a is shown in SEQ ID NO.: 3 or has 1, 2, 3, 4, 5 or more mutations compared to the sequence shown,
the amino acid sequence of the Ig-like C2 type 3 of CD64 is shown in SEQ ID NO.: 5 or has 1, 2, 3, 4, 5 or more mutations compared to the sequence shown,
the amino acid sequence of the extracellular hinge region of CD64 is shown in SEQ ID NO.: 7 or has 1, 2, 3, 4, 5 or more mutations compared to the sequence shown,
the amino acid sequence of the transmembrane region of CD16a is shown in SEQ ID NO.: 9 or has 1, 2, 3, 4, 5 or more mutations compared to the sequence shown,
the amino acid sequence of the intracellular region of CD16a is shown in SEQ ID NO.: 11 or has 1, 2, 3, 4, 5 or more mutations compared to the shown sequence,
the amino acid sequence of the intracellular region of CD64A Ig-like C2 type 1 is shown in SEQ ID NO.: 15 or has 1, 2, 3, 4, 5, or more mutations with the shown sequence, and
the amino acid sequence of the intracellular region of CD64A Ig-like C2 type 2 is shown in SEQ ID NO.: 17 or has 1, 2, 3, 4, 5 or more mutations with the shown sequence.

3. The fusion protein as claimed in claim 1, wherein the fusion protein is coded by a coding nucleic acid.

4. The fusion protein as claimed in claim 3, wherein:
the encoding nucleic acid sequence of Ig-like C2 type 1 of CD16a has 85% or more homology with the sequence shown in SEQ ID NO.: 2, partially or completely complementary with the sequence shown in SEQ ID NO.: 2, or as shown in SEQ ID NO.: 2,
the coding nucleic acid sequence of Ig-like C2 type 2 of CD16a has 85% or more homology with the sequence shown in SEQ ID NO.: 4, or partially or completely complementary with the sequence shown in SEQ ID NO.: 4, or as shown in SEQ ID NO.: 4,
the coding nucleic acid sequence of Ig-like C2 type 3 of CD64 has 85% or more homology with the sequence shown in SEQ ID NO.: 6, or partially or completely complementary with the sequence shown in SEQ ID NO.: 6, or as shown in SEQ ID NO.: 6,
the coding nucleic acid sequence of the extracellular hinge region has 85% or more homology with the sequence shown in SEQ ID NO.: 8, or partially or completely complementary with the sequence shown in SEQ ID NO.: 8, or as shown in SEQ ID NO.: 8,
the coding nucleic acid sequence of the transmembrane region has 85% or more homology with the sequence shown in SEQ ID NO.: 10, or partially or completely complementary with the sequence shown in SEQ ID NO.: 10, or as shown in SEQ ID NO.: 10,
the coding nucleic acid sequence of the intracellular region has 85% or more homology with the sequence shown in SEQ ID NO.: 12, or partially or completely complementary with the sequence shown in SEQ ID NO.: 12, or as shown in SEQ ID NO.: 12,
the coding nucleic acid sequence of CD64A Ig-like C2 type 1 has 85% or more homology with the sequence shown in SEQ ID NO.: 16, or partially or completely complementary with the sequence shown in SEQ ID NO.: 16, or as shown in SEQ ID NO.: 16, and
the coding nucleic acid sequence of CD64A Ig-like C2 type2 has 85% or more homology with the sequence shown in SEQ ID NO.: 18, or partially or completely complementary with the sequence shown in SEQ ID NO.: 18, or as shown in SEQ ID NO.: 18.

5. The fusion protein as claimed in claim 1, wherein the fusion protein is expressed by an expression vector or a host cell.

6. The fusion protein as claimed in claim 5, wherein the expression vector includes but is not limited to bacterial plasmid vector, bacteriophage vector, yeast plasmid vector, adenovirus vector, retrovirus vector, and lentivirus vector.

7. The fusion protein as claimed in claim 5, wherein the host cell includes human immune cells or stem cells.

8. The fusion protein as claimed in claim 7, wherein the immune cells include one or more of T cells, B cells, K cells, and NK cells.

9. The fusion protein as claimed in claim 7, wherein the immune cell is NK cell and the stem cell is iPSC.

10. A method for preparing immune cells with high cytotoxicity, comprising introducing one or more of the fusion protein, the polynucleotide, and the vector into immune cells;
alternatively, the method comprises introducing one or more of the fusion protein, the polynucleotide, and the vector into stem cells, and then inducing stem cells to differentiate into immune cells.

11. The method as claimed in claim 10, wherein the method includes electroporation, protoplast fusion, calcium phosphate precipitation, cell fusion using encapsulated DNA, microinjection, and complete virus transfection.

12. The method as claimed in claim 10, further comprising contacting immune cells with cancer cells in vitro.

13. The method as claimed in claim 12, wherein the cancer cells are prostate cancer cells.

14. An application comprising:
the application of one or more of the fusion protein, the polynucleotide, the vector, and host cell as a pharmaceutical composition;
or, the application of one or more of the fusion protein, the polynucleotide, the vector, the host cell and the pharmaceutical composition in preparation of cancer immunotherapy drugs, autoimmune disease drugs, anti-aging drugs, medical beauty products, and metabolic disease drugs;
or the application of one or more of the fusion protein, the polynucleotide, the vector, the host cell and the pharmaceutical composition in improving the therapeutic effect of monoclonal antibodies.

15. The application as claimed in claim 14, wherein the pharmaceutical composition also contains other drugs for treating cancer and/or structures recognized by the fusion protein.

16. The application as claimed in claim 14, wherein the drug is a monoclonal antibody drug, which includes an antibody with the product number ab268061 provided by abcam company or a marketed monoclonal antibody drug, the marketed monoclonal antibody drug includes Matuximab, Trastuzumab, Cituximab, Dalizumab, Tanizumab, Abavozumab Admuzumab, Aftuzumab, Alenzumab, Peihua Aczumab, Almatuzumab, Abazumab, Paviximab, Betomozumab, Belimuzumab, Bevaczumab, Mobivaczumab, Berentzumab Vititin, Mocantzumab, Lacantzumab, Carolizumab Penditide, Carotozumab, Positazumab, Situxumab, Konamizumab, Dacitazumab Dalozumab, Desmozumab, Emexizumab, Ezuzuzumab, Ezuzuzumab, Ensiximab, Epacizumab, Emasozumab, Adazumab, Falezumab, Fentolumab, Galicizumab, Gizuzumab, Gizuzumab, Giriximab, Gleizumab Vititin, Teimozumab, Igovozumab, Laindacizumab, Intuxumab, Izumab, Ozomicin Epilimumab, Itomumab, Labezumab, Laishamumab, Lintuzumab, or Molovozumab.

17. The application as claimed in claim 14, wherein:
the pharmaceutical composition further comprises pharmaceutically acceptable carriers, diluents, or excipients,
the pharmaceutically acceptable carriers, diluents, or excipients include, but are not limited to, any adjuvants, vectors, excipients, flow aids, sweeteners, diluents, anti-corrosion agents, dyes/colorants, flavor enhancers, surfactants, wetting agents, dispersants, suspensions, stabilizers, isotonic agents, solvents that have been approved by the US Food and Drug Administration or the China Food and Drug Administration for use in humans or livestock Surfactants or emulsifiers, and
the pharmaceutical composition is tablets, pills, powders, granules, capsules, pastilles, syrups, liquids, emulsions, suspensions, controlled release preparations, aerosols, films, injections, intravenous drops, transdermal absorption preparations, ointments, lotions, adhesive preparations, or suppositories.

18. The application as claimed in claim 14, wherein:
the cancers include cervical cancer, seminoma, testicular lymphoma, prostate cancer, ovarian cancer, lung cancer, rectal cancer, breast cancer, skin squamous cell cancer, colon cancer, liver cancer, pancreatic cancer, stomach cancer, esophageal cancer, thyroid cancer, bladder transitional epithelial cancer, leukemia, brain tumor, stomach cancer, peritoneal cancer, head and neck cancer, endometrial cancer, kidney cancer Female reproductive tract cancer, in situ cancer, neurofibroma, bone cancer, skin cancer, gastrointestinal stromal tumor, mast cell tumor, multiple myeloma, melanoma, or glioma,
the autoimmune disease includes achalasia, Addison's disease, adult Steele's disease, agammaglobulinemia, alopecia areata, amyloidosis, ankylosing spondylitis, anti GBM/anti TBM nephritis, antiphospholipid syndrome, autoimmune vascular edema, autoimmune autonomic dysfunction, autoimmune encephalomyelitis, autoimmune hepatitis, autoimmune inner ear disease, autoimmune myocarditis Autoimmune oophoritis, autoimmune orchitis, autoimmune pancreatitis, autoimmune retinopathy, or autoimmune urticaria, and the metabolic diseases include diabetes, diabetes ketoacidosis, hyperglycemia and hypertonic syndrome, hypoglycemia, gout, protein energy malnutrition, vitamin A deficiency, scurvy, vitamin D deficiency or osteoporosis.

19. The application as claimed in claim 14, wherein the therapeutic effect is specific to prostate cancer.


SEQUENCE LISTING (18 sequences)

SEQ ID NO: 1 - protein, 105 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVH

SEQ ID NO: 2 - DNA, 315 bp, synthetic construct
  source 1..315: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccat

SEQ ID NO: 3 - protein, 85 aa, synthetic construct
IGWLLLQAPRWVFKEEDPIHLRCHSWKNTALHKVTYLQNGKGRKYFHHNSDFYIPKATLKDSGSYFCRGLFGSKNVSSETVNITI

SEQ ID NO: 4 - DNA, 255 bp, synthetic construct
  source 1..255: mol_type = other DNA
atcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatc

SEQ ID NO: 5 - protein, 93 aa, synthetic construct
VKELFPAPVLNASVTSPLLEGNLVTLSCETKLLLQRPGLQLYFSFYMGSKTLRGRNTSSEYQILTARREDSGLYWCEAATEDGNVLKRSPELE

SEQ ID NO: 6 - DNA, 279 bp, synthetic construct
  source 1..279: mol_type = other DNA
gtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggag

SEQ ID NO: 7 - protein, 15 aa, synthetic construct
LQVLGLQLPTPVWFH

SEQ ID NO: 8 - DNA, 45 bp, synthetic construct
  source 1..45: mol_type = other DNA
cttcaagtgcttggcctccagttaccaactcctgtctggtttcat

SEQ ID NO: 9 - protein, 21 aa, synthetic construct
VSFCLVMVLLFAVDTGLYFSV

SEQ ID NO: 10 - DNA, 63 bp, synthetic construct
  source 1..63: mol_type = other DNA
gtctctttctgcttggtgatggtactcctttttgcagtggacacaggactatatttctctgtg

SEQ ID NO: 11 - protein, 25 aa, synthetic construct
KTNIRSSTRDWKDHKFKWRKDPQDK

SEQ ID NO: 12 - DNA, 78 bp, synthetic construct
  source 1..78: mol_type = other DNA
aagacaaacattcgaagctcaacaagagactggaaggaccataaatttaaatggagaaaggaccctcaagacaaatga

SEQ ID NO: 13 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFVIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 14 - DNA, 765 bp, synthetic construct
  source 1..765: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttcgttattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 15 - protein, 80 aa, synthetic construct
AVITLQPPWVSVFQEETVTLHCEVLHLPGSSSTQWFLNGTATQTSTPSYRITSASVNDSGEYRCQRGLSGRSDPIQLEIH

SEQ ID NO: 16 - DNA, 240 bp, synthetic construct
  source 1..240: mol_type = other DNA
gcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccac

SEQ ID NO: 17 - protein, 83 aa, synthetic construct
RGWLLLQVSSRVFTEGEPLALRCHAWKDKLVYNVLYYRNGKAFKFFHWNSNLTILKTNISHNGTYHCSGMGKHRYTSAGISVT

SEQ ID NO: 18 - DNA, 249 bp, synthetic construct
  source 1..249: mol_type = other DNA
agaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcact